(19) United States
(12) Patent Application Publication
(10) Pub. No.: US 2022/0157430 A1
(43) Pub. Date: May 19, 2022

(54) Nutrition Intake Calculator

(71) Applicant: Nicole Patricia Victor, Coconut Creek, FL (US)

(72) Inventor: Nicole Patricia Victor, Coconut Creek, FL (US)

(21) Appl. No.: 17/098,969

(22) Filed: Nov. 16, 2020

(51) Int. Cl. G16H 20/60; G16H 50/70; G16H 15/00; G06Q 50/00; G06K 7/14; G16H 10/60; G06F 16/9535; H04L 29/08

(52) U.S. Cl. CPC G16H 20/60; G16H 50/70; G16H 15/00; H04L 67/22; G06K 7/1413; G16H 10/60; G06F 16/9535; G06Q 50/01

(57) ABSTRACT

The Nutrition Intake Calculator App., is invented to scan, calculate and document the information in Food, the Nutrition and Supplement Facts Panel, into a readable format; and then show the daily intake of Nutrients, Vitamins and Minerals, and then compare and contrast them against those needed for recommended daily nutritional needs; and report on whether they are gained, or not.

The Nutrition Intake Calculator App., will offer a search box option for looking up everyday foods, and their nutritional content to enable the input information of individual's recommended daily dose of nutrients, vitamins, and minerals; and the ability to Subscribe, Like and share on all Social Media Platforms; The Nutrition Intake Calculator will be used within the spectrum of an Application (APP) format for Cellular Phone, Tablets, and any other devices able to scan barcodes, and input data for interpretation.

This invention is in reference to application No. 62/956,126

### BACKGROUND OF THE INVENTION

Nutrition Intake Calculator, is invented to be used as an Application (APP) format, through Cellular Phones, Tablets, and any other devices able to scan barcodes. For example, devices on the Android, Apple, and Amazon App. Store.

This works by adding information which came from Food, the Nutrition and Supplement Facts Panel, as data into a readable format.

This invention will document the recommended daily dose of Vitamins and Minerals for individual's optimum health; It will document the Vitamins and Minerals that have been achieved daily through the intake of food, and supplements; then document and calculate whether the recommended daily dose have been achieved, surpassed or not. This invention will help individuals to make informed decisions to better manage their health, with the knowledge of the vitamins, minerals, and Supplements being placed in their bodies.

### BRIEF SUMMARY

The Nutrition Intake Calculator App., is created to be used for Cellular Phones, Tablets, and all other devices able to scan barcodes, and in-put data into a readable format for interpretation.

To begin, the app format will allow for individuals to input personal data, such as: Age; Gender; Weight, and Height, to give personalized readings. Once this information is inputted; it will be used as the primary information each time the Scanner is used, unless deleted.

After, the Nutrition and Supplement Facts Panel's content is scanned, or, the information from foods are inputted; it will give a detailed report on Calories, Vitamins, Minerals, Nutrients, and Macronutrients included. That information will then be calculated in contrast with the recommended daily dose of those Vitamins, Nutrients, and Minerals needed for individual's optimum health.

The next step in the process will address whether or not the information inputted via the Scanner, shows that the user has achieved his/her daily nutritional needs, and if not; which nutritional elements are still missing if any, and how much.

### DETAILED DESCRIPTION

The Nutrition Intake Calculator App., for Food, Nutrition, and Supplement Facts Panel is invented to help individuals make better health choices to manage their health. It will be used within the spectrum of an Application (APP) format for Cellular Phone, Tablets, and any other devices able to scan barcodes, and input data for interpretation.

The Nutrition Intake Calculator App., is invented to scan, document, and calculate the information in Food, the Nutrition and Supplement Facts Panel, into a readable format; which will then show the Nutrients, Vitamins and Minerals needed for recommended daily nutritional needs; and compare and contrast whether they are gained, or not, to help individuals make better health choices, to achieve optimum health.

The Nutrition Intake Calculator App., will have a Search Engine Capability Feature, to look up the nutritional contents in foods to be inputted as data for their nutritional value.

The Nutrition Intake-Calculator App., will offer a list of foods that will help to fight inflammation, lowers blood pressure, shrink tumors, with/without allergens etc.

The Nutrition Intake Calculator App., will be activated by first inputting personal data such as: Age, Gender, Weight, Height, in order to calculate the personal recommended daily dose of Calories, Vitamins, Minerals, Nutrients, and Macronutrients, needed for individual optimum health.

The Nutrition Intake Calculator App., will have the ability to add portion of ¼, ½, ¾, and 1 up to 100 to measure the size of the amount of food being inputted, for interpretation. The Nutrition Intake Calculator App., will give other personalized readings to users, tailored to their individual need. These readings will be based on specific pre-inputted data, such as: Age, Gender, Weight, Height, Body Mass Index, Number Glasses of water per day, Number Glasses of Juice per day; Number days of Exercise per week, which will result in a report detailing Hydration Factor; Weight Loss; Weight Gain; Recommended Weight, Muscle Mass; Total Body Fat, and suggested weight, as data accumulates.

The Nutrition Intake Calculator App., will keep old information, update this report daily, and archived them for continuous use, and reference. The archived information will be kept until a pre-determined amount of time, with the oldest data being deleted first, as new ones are added, to allow users to keep track of their health progress.

This invention will aid in helping individuals to use the information provided to make better health choices, by including foods in their diet, that will satisfy their daily recommended nutritional needs.

The Nutrition Intake Calculator App., will offer a search box option for looking up everyday foods, and their nutritional content to enable the input information of individual's recommended daily dose of nutrients, vitamins, and minerals; The ability to Subscribe, Like and share on all Social Media Platforms; and the ability to delete item/s or information already inputted.

The Nutrition Intake Calculator App., will address the problem of not knowing the value of the Calories, Vitamins, Minerals, Nutrients, and Macronutrients, needed on a daily basis to achieve ones' optimal health. It will then solves that problem by, providing a report of those Vitamins, and Minerals gained through the daily intake of documented nutrients in food and supplements; then report on those still needed, if any; and which group of food essentially provides them, if needed.

This invention also addresses the need for making better health choices, to achieve nutritional balance. It will be most helpful to those seeking to obtain optimum health; those caring for their health; and those needing help to make better health choices, with a more proactive approach.

Following, is a sample of the information, as it would appear in the Application (APP).

### Daily Nutrition Intake—Calculator Application: For Food, Nutrition, and Supplement Fact Panel

### Input Tab

### Personal Information Section

Gender: (Man-Woman)

Age: (0 months-100 Yrs)

Height: (0 ft; 0 inches-10 ft; 11 inches)

Weight: (0-2,000 LBS)

### Result Tab

| | | | |
|---|---|---|---|
| Woman | 44 yrs | 5″ 2′ | 1401b |

Body Mass Index (BMI): 27.7

Estimated Daily Caloric Needs: 2041 kcal/day

### Food Input Section

### Scan Nutritional and Supplemental Fact Panel Barcode

### Input Tab For Foods Not on Nutritional and Supplemental Facts Panel

Example: Potatoe

Quantity: ¼; ½; ¾; 1; 2; 3; 4; . . .

### Calculabe Button

Results shown in: Normal Distribution/ND; Grams/g; Milligrams/mg; Micro grams/mcg

### Results

From
Food &

| Nutri | RC/Dy | | Tolrble/Dy | | Crnt/Day | | |
|---|---|---|---|---|---|---|---|
| Panel | Intake | | Intake | | Intake | | Correct/Missing/over |
| Vitamins: | Calories: | | | | | | |
| Vitamin A | 700 | mcg | 3,000 | mcg | 500 | mcg | No/200 mcg/ |
| Vitamin B₆ | 1.3 | mg | 100 | mcg | 75 | mcg | |
| Vitamin B_{12.} | 2.4 | mcg | | | | | |
| Vitamin C | 75 | mg. | 2,000 | mg | 50 | mg | /25 mg/ |
| Vitamin D | 15 | mcg | 100 | mcg | 20 | mcg | Yes//5 mcg |
| Vitamin E | 15 | mg | 1,000 | mg | | | |
| Vitamin K | 90 | mcg | ND | | 80 | mcg | No/10 mcg/ |
| Thiamin | | | | | | | |
| Foliate | | | | | | | |
| Niacin | | | | | | | |
| Riboflavin | | | | | | | |
| Choline | | | | | | | |
| Pantothenic- | | | | | | | |
| Acid | | | | | | | |
| Biotin | | | | | | | |

Minerals:

Magnesium

Manganese

Molybdenum

Phosphorus

Potassium

Chromium

Selenium

Calcium

Chloride

Fluoride

Copper

Sodium

Iodine

Iron

Zinc

Macronutrients:

Carbohydrate

Saturated Fat

Cholesterol

Trans Fat

Protein

Sugars

Fiber

### Results

| From | | | | | | | |
|---|---|---|---|---|---|---|---|
| Supple | RC/Dy | | Tolrble/Dy | | Crnt/Day | | |
| Panel | Intake | | Intake | | Intake | | Correct/Missing/over |
| Vitamins: | Calories: | | | | | | |
| Vitamin A | 700 | mcg | 3,000 | mcg | 500 | mcg | No/200 mcg/ |
| Vitamin B₆ | 1.3 | mg | 100 | mcg | 75 | mcg | |
| Vitamin B_{12.} | 2.4 | mcg | | | | | |
| Vitamin C | 75 | mg. | 2,000 | mg | 50 | mg | /25 mg/ |
| Vitamin D | 15 | mcg | 100 | mcg | 20 | mcg | Yes//5 mcg |
| Vitamin E | 15 | mg | 1,000 | mg | | | |
| Vitamin K | 90 | mcg | ND | | 80 | mcg | No/10 mcg/ |
| Thiamin | | | | | | | |
| Foliate | | | | | | | |
| Niacin | | | | | | | |
| Riboflavin | | | | | | | |
| Choline | | | | | | | |
| Pantothenic- | | | | | | | |
| Acid | | | | | | | |
| Biotin | | | | | | | |

Minerals:

Magnesium

Manganese

Molybdenum

Phosphorus

Potassium

Chromium

Selenium

1. The Nutrition Intake Calculator App., is invented to scan, calculate and document the information in Food, the Nutrition and Supplement Facts Panel, into a readable format; and then show the daily intake of Nutrients, Vitamins and Minerals, and then compare and contrast them against those needed for recommended daily nutritional needs; and report on whether they are gained, or not.
The Nutrition Intake Calculator App., will offer a search box option for looking up everyday foods, and their nutritional content to enable the input information of individual's recommended daily dose of nutrients, vitamins, and minerals; and the ability to Subscribe, Like and share on all Social Media Platforms;
The Nutrition Intake Calculator will be used within the spectrum of an Application (APP) format for Cellular Phone, Tablets, and any other devices able to scan barcodes, and input data for interpretation.
